SEQUENCE LISTING (6 sequences)

SEQ ID NO: 1 - protein, 9 aa, Artificial Sequence
  synthetic destruction box motif targeting signal
RXALGXXXN

SEQ ID NO: 2 - protein, 30 aa, Artificial Sequence
  synthetic beta-catenin destabilization sequence, amino acids 18-47
RKAAVSHWQQQSYLDSGIHSGATTTAPSLS

SEQ ID NO: 3 - protein, 102 aa, Artificial Sequence
  synthetic murine IP10 signal peptide linked to mature human MCP3 via a linker
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKL

SEQ ID NO: 4 - protein, 97 aa, Artificial Sequence
  synthetic human IP10 signal peptide linked to mature human MCP3
MNQTAILICCLIFLTLSGIQGQPVGINTSTTCCYRFINKKIPKQRLESYRRTTSSHCPREAVIFKTKLDKEICADPTQKWVQDFMKHLDKKTQTPKL

SEQ ID NO: 5 - protein, 99 aa, Artificial Sequence
  synthetic human MCP3 signal peptide linked to mature human MCP3
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 6 - protein, 35 aa, Artificial Sequence
  synthetic tissue plasminogen activator (tPA) signal peptide and propeptide sequences, tPA secretory signal
MDAMKRGLCCVLLLCGAVFVSPSQEIHARFRRGAR

(19) United States
(12) Patent Application Publication
(10) Pub. No.: US 2013/0052221 A1
(43) Pub. Date: Feb. 28, 2013

(54) DNA-PROTEIN VACCINATION PROTOCOLS

(71) Applicants: Barbara K. Felber, Rockville, MD (US); George N. Pavlakis, Rockville, MD (US)

(72) Inventors: Barbara K. Felber, Rockville, MD (US); George N. Pavlakis, Rockville, MD (US)

(73) Assignee: The Govt. of the U.S, as represented by The Sec. of The Dept. of Health and Human Services, Rockville, MD (US)

(21) Appl. No.: 13/580,892

(22) PCT Filed: Feb. 25, 2011
(86) PCT No.: PCT/US2011/026325

§ 371 Date: Oct. 12, 2012

Related U.S. Application Data
(60) Provisional application No. 61/308,853, filed on Feb. 26, 2010.

(51) Int. Cl. A61K 39/00; A61M 37/00; A61P 31/18; A61K 39/21; A61P 37/04

(52) U.S. Cl. 424/208.1; 424/184.1; 604/501

(57) ABSTRACT

This invention provides a method of co-delivery of combination DNA and protein immunogenic compositions to enhance protective or therapeutic effects.

### CROSS-REFERENCE TO RELATED APPLICATIONS

This application is a U.S. National Phase of PCT/US2011/026325, filed Feb. 25, 2011, which claims benefit of U.S. provisional application No. 61/308,853, filed Feb. 26, 2010, the entire contents of which are incorporated by reference herein in their entirety.

### REFERENCE TO SEQUENCE LISTING

This application includes a Sequence Listing as a text file named “SEQTXT_{—}77867-580100US-845935.txt” created Aug. 20, 2012, and containing 4,866 bytes. The material contained in this text file is incorporated by reference in its entirety for all purposes.

### BACKGROUND OF THE INVENTION

The administration of nucleic acid-based vaccines, including both naked DNA and viral-based vaccines, has been described for the treatment or prevention of cancer or a pathogenic infection. In some embodiments, such vaccines have been developed to treat an individual with a retrovirus infection such as HIV infection. Further, the administration of DNA vaccines in prime boost protocols has been suggested in the prior art (see, e.g., US application no. 2004/033237; Hel et al., J. Immunol. 169:4778-4787, 2002; Barnett et al., AIDS Res. and Human Retroviruses Volume 14, Supplement 3, 1998, pp. S-299-S-309 and Girard et al., C R Acad. Sci. III 322:959-966, 1999 for reviews). However, there is a need for improved vaccination protocols. This invention addresses that need.

### BRIEF SUMMARY OF THE INVENTION

The invention is based, in part, on the discovery that co-administration of a nucleic acid vaccine with a protein vaccine for the same antigen of interest that is encoded by the DNA vaccine provides for improved immune responses relative to protocols that involve prime boost strategies where a DNA vaccine is administered and then followed, typically weeks later, by administration of a protein vaccine.

The invention thus provides a method of generating an immune response in an individual, preferably a human, comprising co-administering a nucleic acid component and a protein component, often at the same site. The protein component comprises a polypeptide of an antigen of interest. The nucleic acid component comprises at least one nucleic acid encoding the antigen of interest. The antigen of interest can be any antigen for which it is desirable to elicit an immune response. This includes cancer antigens as well as antigens from infectious agents, e.g., viruses or other pathogenic organisms.

In some embodiments, the nucleic acid and protein components of an immunization protocol of the invention are co-administered in a naïve individual for the purpose of developing protective immune response. In some embodiments, the components are co-administered to an individual infected with a pathogenic agent, e.g., a virus, to achieve a therapeutic effect. In some embodiments, the individual that is treated in accordance with the methods of the invention is infected with a retrovirus, e.g., HIV. The nucleic acid and protein components can be administered repeatedly. In some embodiments, the nucleic acid component comprises multiple expression vectors. In some embodiments, the co-administration protocols of the invention provide an enhanced antibody response, e.g., superior longevity and/or production of enhanced amounts of antibody measured at a particular time point, compared to prime-boost protocols. In some embodiments, the co-administration protocols of the invention provide superior immunological memory in comparison to prime boost protocols. In typical embodiments, both superior immunological memory and enhanced antibody responses are achieved using the methods of the invention compared to prime boost strategies.

In some embodiments, a nucleic acid component for use in an immunization method of the invention comprises one or more vectors that encode HIV proteins, including a gag protein targeted for secretion, a gag protein targeted for degradation, an env protein targeted for secretion, an env protein targeted for degration, a Pol, Nef, Tat, Vif protein targeted for degradation, a Pol, Nef, Tat protein targeted for degradation and an IL-12 adjuvant.

In some embodiments, the nucleic acid component comprises one or more of the following vectors: Gag expression vectors 2S CATEDX and 21S MCP3p39; Env expression vectors 72S CATEenv and 73S MCP3-Env; Pol Nef Tat Vif and protein expression vectors 44S CATE-PolNTV and 155S CATE-PolNT; and the protein component comprises inactived HIV particles. In some embodiments, the method of the invention further comprises administering a nucleic acid encoding IL-12. The expression vectors 2S CATEDX and 21S MCP3p39; Env expression vectors 72S CATEenv and 73S MCP3-Env; Pol Nef Tat Vif and protein expression vectors 44S CATE-PolNTV and 155S CATE-PolNT are described in Rosati, et al., PNAS, 106:15831-15836 (2009).

In some embodiments, the nucleic acid component comprises one or more vectors that encode a gag protein a secreted gag protein, an env protein, a secreted pol protein, a pol protein targeted for degradation and a nef, tat, vif protein targed for degradation. In some embodiments, the nucleic acid componenet further comprises an expression vector encoding an adjuvant, e.g., IL-12. In some embodiments, the nucleic acid component comprises at least one of the following vectors: 206S gag, 209S MCP3gag p39, 99S Env239, 216S MCP3-pol, 103S LAMP-pol, 147S LAMP-NTV (nef tat vif), and a vector encoding IL-12. The expression vectors 2S CATEDX and 21S MCP3p39; Env expression vectors 72S CATEenv and 73S MCP3-Env; Pol Nef Tat Vif and protein expression vectors 44S CATE-PolNTV and 155S CATE-PolNT are described in Rosati, et al., PNAS, 106:15831-15836 (2009).

In some embodiments, the protein component is administered as virus particles or pseudovirus particles, or together with adjuvants. In some embodiments, the protein component may be administered as a soluble peptide, or may be administered in a formulation that promotes delivery, e.g., liposomes or other formulations.

In some embodiments, an individual treated with a co-immunization method of the invention is a cancer patient, e.g., an individual that has breast cancer, colorectal cancer, lung cancer, prostate cancer, pancreatic cancer, ovarian cancer, or melanoma, where the antigen of interest is a cancer antigen.

In some embodiments, an individual treated with a co-immunization method of the invention may be immunologically naïve with respect of an antigen of interest, e.g., an antigen from a pathogen. Such immunologically naïve individuals have not been previously exposed to the antigen such that an immune response was generated.

### BRIEF DESCRIPTION OF THE DRAWINGS

FIG. 1 is a schematic depicting an example of a vaccination strategy using DNA+protein (AT-2 inactivated viral particles). A group of 8 Indian macaques were vaccinated with DNA alone, and compared to two animals co-immunized with DNA+protein (in the form of AT-2 inactivated viral particles).

FIG. 2 shows rapid and stable Ab generation after DNA-protein co-immunization. Two animals were immunized 2× with DNA+protein and boosted by DNA electroporation (EP). Initial vaccination was IM in the same site for DNA and protein with no adjuvants. The animals were rested for 7 months and then boosted by DNA only and compared to animals (in gray) vaccinated by DNA only. Interestingly, the DNA+protein animals developed high Ab responses from the start, and they also exhibited cell mediated immune response (CML, see below). Thus, DNA+protein vaccination appears to elicit faster and better B cell memory. Further, the antibody response is persistent in the DNA+protein vaccinated animals, compared to the response in the animals receiving DNA only.

FIG. 3 provides data showing that durable neutralizing antibodies were detected in DNA+protein (AT-2 particles) co-immunized animals compared to DNA-only vaccinated animals. Experimental procedures and assay details have been reported in Rosati, et al., PNAS, 106:15831-15836 (2009).

FIG. 4 provides data showing that DNA+protein (AT-2 particles) co-immunized animals had low peak viremia after highly pathogenic SIVmac₂₅₁ challenge. Immunized macaques were challenged via the mucosal route. Plasma virus loads were monitored over time.

FIG. 5 is a schematic depicting a comparison of 3 groups of 8 macaques vaccinated as follows: Group1 received 4 DNA vaccinations by electroporation at the indicated times (0, 8, 16 and 36 weeks). Group2 received DNA+protein (inactivated AT-2 particles) vaccination at the same times. Group3 received 2 DNA vaccinations (weeks 0, 8) followed by inactivated AT-2 particle boost (16 and 36 weeks).

FIG. 6 provides data demonstrating that co-administration of DNA and protein (AT-2 SIVmac239 particles) increased humoral immune responses to env in macaques. Co-delivery of DNA and AT-2 SIVmac239 particles increased Env responses; the effect on Gag response was not significant.

FIG. 7 shows development of cellular immune responses in the 3 groups of animals described in FIGS. 5 and 6, above. Cellular immune responses in the lungs of vaccinated animals were evaluated after bronchioalveolar lavage (BAL) of four animals per group, which were positive for the MamuA*01 haplotype. Celular immune responses against the Gag were determined by Gag tetramer staining Analysis of the gag responses showed that DNA boosts cellular immune responses every time after vaccination (group 1 and 2) and that the protein alone (group 3) did not boost the cellular immune response after the 3rd vaccination.

FIG. 8 shows anti-Env Ab titers (reciprocal end-point dilution, log-scale) of three groups consisting of eight animals per group. Each group was vaccinated with either DNA only, DNA and AT2 particles in the same site, or DNA alone (2×) followed by AT2 particle boost (2×). The analysis was performed two weeks after the third vaccination (week 18). DNA and AT2 particle co-immunization in the same site gives higher Ab levels. The results of Group 2 are superior to Group 1 (DNA immunization) (p=0.0002, Kruskal-Wallis). Reciprocal end-point dilutions were determined by an Env-specific Elisa.

FIG. 9 is a comparison of binding Ab levels for env (top, same as FIG. 6) to Neutralizing Ab (Nab) titers to lab-adapted SIVmac251 (bottom). Group 2 developed maximal Nab titers after 2 vaccinations. The other groups developed lower and less durable Nab.

FIG. 10 provides data demonstrating that animals co-immunized with DNA and different Env protein formulations, using purified HIV env protein together with an adjuvant, had higher antibody titers.

FIG. 11 provides data demonstrating that animals co-immunized with DNA and different env protein formuations not only had binding Abs (FIG. 10), but also developed heterologous neutralizing Abs.

### DETAILED DESCRIPTION OF THE INVENTION

### Definitions

In the context of this invention, a “nucleic acid component” of an immunogenic composition as described herein refers to a component that includes one or more expression vectors that enter cells and are expressed. Typically, multiple vectors that encode the antigen of interest are employed. The term “nucleic acid component” thus includes embodiments in which multiple vectors are administered separately (i.e., the vectors are in separate pharmaceutically acceptable solutions) as well as embodiments in which multiple vectors are administered together in the same solution. The terms refers to both nucleic acids administered as a purified form, e.g., an expression plasmid, as well as nucleic acids that are administered as a virus (within a viral capsid). The nucleic acid encodes antigenic epitopes from an antigen of interest that induce humoral and/or cellular immune responses. The nucleic acid component can be any nucleic acid, including DNA or RNA. For example, in some embodiments, the nucleic acid component can be a viral RNA or a messenger RNA. The nucleic acid encodes immunogenic epitopes of an antigen of interest and stimulates a cellular and/or humoral immune response.

A “protein component” of an immunogenic composition as described herein refers to an antigen that is delivered in a protein form. The protein form can be part of an antigen, e.g., a peptide or fragment of the protein, or may be comprised by other proteins, e.g., may take the form of inactivated viral particles. A “protein component that comprises the antigen of interest” can also comprise antigenic variants of the antigen of interest, e.g., where the antigen of interest is an HIV antigen, a “protein component” can also contain multiple forms of the HIV antigen from different HIV strains. The “protein component” comprises immunogenic epitopes of an antigen of interest and stimulates a cellular and/or humoral immune response.

The terms “enhanced immune response” or “increased immune response” as used herein refers to an immune response to a nucleic acid component and protein component that are co-delivered, where the immune response is increased in comparison to when the nucleic acid component and protein component are administered sequentially with a time frame of from over two weeks, typically from one to two months, separating administration of the nucleic acid and protein components. An “enhanced immune response” may include increases in the level of immune cell activation and/or an increase in the duration of the response and/or immunological memory as well as an improvement in the kinetics of the immune response. The increase can be demonstrated by either a numerical increase, e.g., an increased in levels of antibody in a particular time frame, as assessed in an assay to measure the response assay or by prolonged longevity of the response.

In the context of this invention “co-administration” or “co-delivery” or “co-immunization” refers to administering nucleic acid and protein components at essentially the same time. “At essentially the same time” refers to administering the components within 48 hours of one another, typically within 24 hours of one another, and most often within 12 hours, 6 hours, or 1 hour of one another. In many embodiments, the components are administered within minutes of one another, e.g., within 1 or 10-30 minutes of one another, or are administered at the same time. “Co-administration” includes embodiments in which the protein and nucleic acid components are administered as separate formulations as well as embodiments in which the two components are mixed for administration to the individual.

“Administration at the same site” as used here, typically refers to administering components of a vaccine to a site that is substantially the same site. “Substantially the same site” refers to administration of both components to the same individual. “Administration at substantially the same site” may thus encompass different sites of administration in one individual, e.g., administration of a component intramuscularly to one location, e.g., an arm, and administration of another component intramuscularly to a different location, e.g., the other arm, at the same time. In some embodiments, the components are administered to the same location such as the same limb, e.g., the same arm or the same area of the arm, e.g., the upper arm. “Administration at the same site” or “substantially the same site” may also encompass different routes of administration, e.g., administration of one component, e.g., the nucleic acid component, intramuscularly to a location and another component, e.g., a protein component, intradermally to that same location. This constitutes “administration at the same site” for the purposes of this invention, even though one component is delivered to the muscle and the other component is delivered in the skin. Administration can be achieved in various ways, including but not limited to oral, buccal, sublingual, parenteral, intravenous, intradermal, subcutaneous, intramuscular, transdermal, transmucosal, intranasal, rectal, intrarectal, etc., administration. Administration can be local or systemic.

The terms “treating” and “treatment” refer to delaying the onset of, retarding or reversing the progress of, or alleviating or preventing either the disease or condition to which the term applies, or one or more symptoms of such disease or condition.

An “antigen” refers to a molecule, typically a protein molecule in the current invention, containing one or more epitopes (either linear, conformational or both) that will stimulate a host's immune system to make a humoral and/or cellular antigen-specific response. The term is used interchangeably with the term “immunogen.” Normally, an epitope will comprise between about 7 and 15 amino acids, such as, 9, 10, 12 or 15 amino acids. The term “antigen” includes both subunit antigens, (i.e., antigens which are separate and discrete from a whole organism with which the antigen is associated in nature), as well as inactivated organisms, such as viruses.

In the context of this invention, an “immunologically naïve” individual is an individual who has not been exposed to an antigen of interest. Exposure can be measured using any of a number of known assays, including measurement of antibodies to the antigen of interest or measurements of cellular immune responses such as skin sensitivity test, lymphocyte proliferation assays, or measurements of lymphocyte activation after antigen stimulation.

The term “nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term “nucleic acid” is used interchangeably with gene, cDNA, mRNA, oligonucleotide, and polynucleotide.

The terms “peptide,” “polypeptide,” and “protein” are used interchangeably herein to refer to at least two amino acids or amino acid analogs that are covalently linked by a peptide bond or an analog of a peptide bond. The amino acids of the peptide may be L-amino acids or D-amino acids. A peptide, polypeptide or protein may be synthetic, recombinant or naturally occurring. A synthetic peptide is a peptide produced by artificial means in vitro.

“Conservatively modified variants” as used herein applies to amino acid sequences. One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups are examples that each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region of an antigen of interest or a nucleic acid encoding an antigen of interest when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or can be applied to, the complement of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of a reference amino acid or reference nucleic acid sequence.

For sequence comparison, typically one sequence acts as a reference sequence, to which test sequences are compared. A preferred example of an algorithm that is suitable for determining percent sequence identity and sequence similarity are the BLAST algorithms, which are described in Altschul et al., Nuc. Acids Res. 25:3389-3402 (1977) and Altschul et al., J. Mol. Biol. 215:403-410 (1990), respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

An “adjuvant” in the context of this invention refers to a composition that enhances the immune response. A “molecular adjuvant” in the context of this invention refers to a biologically produced molecule, often a recombinant polypeptide, that enhances the immune response. These include molecules such as IL-2, IL-12, and the IL-15 and IL-15Receptor alpha combination, which are often administered by injection in the form of DNA plasmids that express the bioactive molecules in vivo.

A “pharmaceutical excipient” comprises a material such as a carrier, pH-adjusting and buffering agents, tonicity adjusting agents, wetting agents, preservative, and the like.

“Pharmaceutically acceptable” refers to a non-toxic, inert, and/or composition that is physiologically compatible with humans or other mammals.

### Introduction

This invention is based on the discovery that a combination of a nucleic acid vaccine encoding an antigen of interest and protein vaccine that comprises the antigen, when co-administered, e.g., at the same site, results in an enhanced immune response, e.g., superior immunological memory, including superior longevity of antibody response, in comparison to a prime/boost strategy in which a DNA priming vaccine is followed by administration of DNA as a boosting vaccine or administration of a protein boosting vaccine. In the co-adminsitration protocols of the invention, the nucleic acid and protein components are administered at substantially the same time. Although prime boost strategies have been employed in the past, such strategies do not administer a nucleic acid component and protein component together for the initial immunization of the subject.

By combining the biosynthetically produced, i.e., protein expressed from the administered nucleic acid component, and the exogenously produced antigen, i.e., the protein component, one observes results that are superior to the individual or sequential administration of the same immunogens. The combination induces optimal levels of cellular and humoral immune response resulting in higher, longer and more effective immunological response.

An antigen of interest may be any antigen to which it is desirable to elicit an immune response, e.g., a tumor antigen or an antigen from a pathogenic organism. In one aspect of the invention, the antigen of interest is an HIV antigen, e.g., an HIV env antigen and/or an HIV gag antigen and/or an HIV pol, nef, tat, or vif antigen.

### Components

### Nucleic Acid Component

A nucleic acid component(s) of a combination nucleic acid/protein vaccine of the invention encodes an antigen of interest to which it is desirable to elicit an immune response. Often, the nucleic acid component(s) is one or more purified nucleic acid molecules, for example, one or more plasmid-based vectors (“naked” DNA). In some embodiments, the antigen of interest is encoded by different expression cassettes that produce one or more forms of the antigen that are targeted to the secretion pathway or targeted for degradation. Multiple forms of the antigen may be encoded by a single vector, but are often encoded by multiple vectors. In some embodiments, the nucleic acids are mixed together as a cocktail and administered. In other embodiments, the nucleic acids are maintained as separate formulations.

In some embodiments, the nucleic acid component may comprise vectors that encode the antigen of interest where the vector is contained within a virus. Viral delivery systems include adenovirus vectors, adeno-associated viral (AAV) vectors, herpes viral vectors, retroviral vectors, poxyiral vectors, or lentiviral vectors. Methods of constructing and using such vectors are well known in the art.

Recombinant viruses in the pox family of viruses can be used for delivering the nucleic acid molecules encoding the antigens of interest. These include vaccinia viruses and avian poxviruses, such as the fowlpox and canarypox viruses. Methods for producing recombinant pox viruses are known in the art and employ genetic recombination. See, e.g., WO 91/12882; WO 89/03429; and WO 92/03545. A detailed review of this technology is found in U.S. Pat. No. 5,863,542. Representative examples of recombinant pox viruses include ALVAC, TROVAC, and NYVAC.

A number of adenovirus vectors have also been described that can be used to deliver one or more of the nucleic acid components of the vaccine. (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Retroviruses also provide a platform for gene delivery systems. A number of retroviral systems have been described (U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis, Semliki Forest, and Venezuelan Equine Encephalitis viruses, can also be used as viral vectors to deliver one or more nucleic acid components of the nucleic acid/protein combination vaccines of the invention. For a description of Sindbis-virus derived vectors useful for the practice of the instant methods, see, Dubensky et al., J. Virol. (1996) 70:508-519; and International Publication Nos. WO 95/07995 and WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998).

As noted above, the nucleic acid component(s) of the invention can include embodiments in which vectors encode one or more forms of the antigen of interest for which it is desired to produce an immune response. Such embodiments typically results in enhanced immune responses in comparison to embodiments in which only one form of the antigen is used.

DNA immunization plasmids have been developed that encode fusion proteins that contain a destabilizing amino acid sequence attached to a polypeptide sequence of interest that when administered with a nucleic acid encoding a secreted fusion protein containing a secretory peptide attached to a polypeptide of interest, enhances the immune response (see. e.g., WO 200236806). Combinations of such DNA immunization plasmids have been administered to animals that have undergone antiretroviral therapy (WO2006 010106). WO 2008/089144 also teaches combinations of vectors that encode different form of an antigen for eliciting immune responses to lentiviral antigens.

### Expression Vectors Encoding Fusion Polypeptides Comprising a Degradation Signal

A “destabilizing amino acid sequence” or “destabilization sequence” as used herein refers to a sequence that targets a protein for degradation in the ubiquitin proteosome pathway. Such sequences are well known in the art. Examples of sequences are described, e.g., in WO 02/36806 and WO 2008/089144. A destabilizing sequence that is fused to an antigen of interest comprises the region of the molecule from which the destabilizing sequence is obtained that mediates interaction with the ubiquitin proteosome sequence.

### Targeting to the Proteasome and Other Degradation Signals

A variety of sequence elements confer short lifetime on cellular proteins due to proteasomal degradation and are known in the art. Such sequences can be joined to an antigen of interest that is encoded by a nucleic acid component for use in the invention.

One example of destabilizing sequences are so-called PEST sequences, which are abundant in the amino acids Pro, Asp, Glu, Ser, Thr (they need not be in a particular order), and can occur in internal positions in a protein sequence. A number of proteins reported to have PEST sequence elements are rapidly targeted to the 26S proteasome. A PEST sequence typically correlates with a) predicted surface exposed loops or turns and b) serine phosphorylation sites, e.g. the motif S/TP is the target site for cyclin dependent kinases.

Additional destabilization sequences relate to sequences present in the N-terminal region. In particular the rate of ubiquitination, which targets proteins for degradation by the 26S proteasome can be influenced by the identity of the N-terminal residue of the protein. Thus, destabilization sequences can also comprise such N-terminal residues, “N-end rule” targeting (see, e.g., Tobery et al., J. Exp. Med. 185:909-920).

Other targeting signals include the destruction box sequence that is present, e.g., in cyclins. Such a destruction box has a motif of 9 amino acids, R1(A/T)2(A)3L4(G)5×6(I/V)7(G/T)8(N)9 (SEQ ID NO:1), in which the only invariable residues are R and L in positions 1 and 4, respectively. The residues shown in brackets occur in most destruction sequences. (see, e.g., Hershko & Ciechanover, Annu Rev. Biochem. 67:425-79, 1998). In other instances, destabilization sequences lead to phosphorylation of a protein at a serine residue (e.g., IKba).

Additional degradation signals that can be used to modify an antigen of the invention, e.g., a retroviral antigen such as an HIV or SIV antigen include the F-box degradation signal, such as the F-BOX signal 47aa (182-228) from protein beta-TrCP (Liu, et al., Biochem Biophys Res Comm. 313:1023-1029, 2004). Accordingly, in some embodiments, an expression vector for use in the invention may encode a fusion protein where an F-box degradation signal is attached to an antigen, e.g., an HIV antigen such as gag, pol, env, nef, tat, and/or vif.

### Lysosomal Targeting Sequence

In other embodiments, signals that target proteins to the lysosome may also be employed in the nucleic acid constructs encoding the antigen of interest for use in the co-administration methods of the invention. For example, the lysosome associated membrane proteins1 and 2 (LAMP-1 and LAMP-2) include a region that targets proteins to the lysosome. Examples of lysosome targeting sequences are provided, e.g., in U.S. Pat. Nos. 5,633,234; 6,248,565; and 6,294,378.

As explained above, destabilizing sequences present in particular proteins are well known in the art. Exemplary destabilization sequences include 13-Catenin; and fragments and variants, of those segments that mediate destabilization. Such fragments can be identified using methodology well known in the art. For example, polypeptide half-life can be determined by a pulse-chase assay that detects the amount of polypeptide that is present over a time course using an antibody to the polypeptide, or to a tag linked to the polypeptide. Exemplary assays are described, e.g., in WO02/36806, which is incorporated by reference.

An example of a of β-catenin destabilization sequence (amino acids 18-47) employed in the examples is: RKAAVSHWQQQSYLDSGIHSGATTTAPSLS (SEQ ID NO:2).

Variants of degradation sequences, e.g., that have at least 90% identity, usually at least 91%, 92%, 93%, 94%, 95%, 96%, 97%, 98%, 99%, or greater, identity to a reference sequence, e.g., a reference β-catenin (18-47) sequence, can be employed in this invention.

Expression Vectors that Encode Secreted Fusion Proteins

The nucleic acid components of the invention (e.g., plasmid DNA or viral vector-based nucleic acid components) also typically comprise expression units that encode a fusion protein that includes a secretory polypeptide. A secretory polypeptide in the context of this invention is a polypeptide signal sequence that results in secretion of the protein to which it is attached. In some embodiments, the secretory polypeptide that results in secretion is a chemokine, cytokine, or lymphokine, or a fragment of the chemokine, cytokine, or lymphokine that retains immunostimulatory activity. Examples of secretory polypeptides include chemokines such as MCP-3 or IP-10, or cytokines such as GM-CSF, IL-4, or IL-2. Constructs encoding secretory fusion proteins are disclosed, e.g., in WO02/36806 and WO 2008/089144.

Many secretory signal peptides are known in the art and can be determined using methods that are conventional in the art. For example, in addition to chemokines, secretory signals such as those from tissue plasminogen activator (tPA) protein, growth hormone, GM-CSF, and immunoglobulin proteins may be used. Constructs encoding secretory fusion proteins are disclosed, e.g., in WO02/36806 and WO 2008/089144.

In some embodiments, a secretory signal for use in the invention is MCP-3 amino acids 33-109, e.g., linked to IP-10 secretory peptide. Variants of such sequences, e.g., that have at least 90% identity, usually at least 91%, 92%, 93%, 94%, 95%, 96%, 97%, 98%, 99%, or greater, identity to MCP-3 and/or IP-10, can be employed in this invention.

An example of an IP10 sequence linked to MCP3 that could be used is:

The combination of the murine IP10 linked to the mature human MCP3 (SEQ ID NO:3):

M N P S A A V I F C L I L L G L S G T Q G
(murine IP10 signal peptide)
I L D M A
(linker)
Q P V G I N T S T T C C Y R F I N K K I P K Q R L E S Y R R T T S S H
C P R E A V I F K T K L D K E I C A D P T Q K W V Q D F M K H L D
K K T Q T P K L
(mature human MCP3)

The combination of the human IP10 linked to the mature human MCP3 (SEQ ID NO:4):

M N Q T A I L I C C L I F L T L S G I Q G
(human IP10 signal peptide)
Q P V G I N T S T T C C Y R F I N K K I P K Q R L E S Y R R T T S S H
C P R E A V I F K T K L D K E I C A D P T Q K W V Q D F M K H L D
K K T Q T P K L
(mature human MCP3)

An alternative the human MCP3 using its own signal peptide is used (SEQ ID NO:5):

M K A S A A L L C L L L T A A A F S P Q G L A
(human MCP-3 signal peptide)
Q P V G I N T S T T C C Y R F I N K K I P K Q R L E S Y R R T T S S H
C P R E A V I F K T K L D K E I C A D P T Q K W V Q D F M K H L D
K K T Q T P K L
(mature human MCP3)

In other embodiments, tissue plasminogen activator signal peptide and propeptide sequences are known in the art (see, Delogu, et al, Infect Immun (2002) 70:292; GenBank Accession No. E08757). In some embodiments, the tPA secretory signal is SEQ ID NO:6):

M D A M K R G L C C V L L L C G A V F V S P
(tPA signal aa 1-22)
S Q E I H A R F R R G A R
(tPA propeptide aa 23-35)

Nucleic acids expression cassettes encoding antigens of interest, such as the antigens described above, e.g., antigens modified to be targeted for secretion or degradation, can also be employed with expression cassettes encoding unmodified antigen.

### Expression of Nucleic Acids

In typical embodiments, the nucleic acids encoding the polypeptides, e.g., HIV env, gag, etc. are engineered to removed inhibitor sequences, e.g., by codon substitution; or otherwise codon optimized for expression in the subject treated in accordance with the methods of the invention. See, e.g., U.S. Pat. No. 6,602,705 and International Publications WO 00/39302; WO 02/04493; WO 00/39303; and WO 00/39304 for examples of HIV-encoding polynucleotides that have inhibitory sequences removed. Examples of such engineered sequences are also described in WO 2008/089144.

A nucleic acid component used in the methods of the invention can be administered as one or more constructs. In some embodiments, the protein(s) encoded by the nucleic acid component can comprise an antigen that contains multiple polypeptides, e.g., multiple HIV structural and/or regulatory polypeptides or immunogenic epitopes thereof, where the proteins are encloded by a single expression vector. In other embodiments, the proteins are encoded by multiple expression vectors, or as one or more expression vectors encoding multiple expression units, e.g., a discistronic, or otherwise multicistronic, expression vectors.

Within each expression cassette, sequences encoding an antigen for use in the nucleic acid vaccines of the invention will be operably linked to expression regulating sequences. “Operably linked” sequences include both expression control sequences that are contiguous with the nucleic acid of interest and expression control sequences that act in trans or at a distance to control the gene of interest. Expression control sequences include appropriate transcription initiation, termination, promoter and enhancer sequences; efficient RNA processing signals such as splicing and polyadenylation signals; sequences that stabilize cytoplasmic mRNA; sequences that promote RNA export (e.g., a constitutive transport element (CTE), a RNA transport element (RTE), or combinations thereof; sequences that enhance translation efficiency (e.g., Kozak consensus sequence); sequences that enhance protein stability; and when desired, sequences that enhance protein secretion.

Any of the conventional vectors used for expression in eukaryotic cells may be used for directly introducing nucleic acids into tissue. Expression vectors containing regulatory elements from eukaryotic viruses are often used in eukaryotic expression vectors. Such regulatory elements include, e.g., human CMV, simian CMV, viral LTRs, and the like. Typical vectors may comprise, e.g., those with a human CMV promoter, bovine growth hormone polyA site and an antibiotic resistance gene for selective growth in bacteria.

In some embodiments, the nucleic acid sequences that encode the polypeptides to be expressed are operably linked to one or more mRNA export sequences. Examples include the constitutive transport element (CTE), which is important for the nucleo-cytoplasmic export of the unspliced RNA of the simian type D retroviruses. Another exemplified RNA export element includes the RNA transport element (RTE), which is present in a subset of rodent intracisternal A particle retroelements. The CTE and RTE elements can be used individually or in combination.

Other expression vector components are well known in the art, including, but not limited to, the following: transcription enhancer elements, transcription termination signals, polyadenylation sequences, splice sites, sequences for optimization of initiation of translation, and translation termination sequences.

In some embodiments, the nucleic acid component may comprises one or more RNA molecules, such as viral RNA molecules or mRNA molecules that encode the antigen of interest.

In some embodiments where HIV antigens are employed, expression cosntructs may also contain Rev-independent fragments of genes that retain the desired function (e.g., for antigenicity of Gag or Pol, particle formation (Gag) or enzymatic activity (Pol)), or may also contain Rev-independent variants that have been mutated such that the encoded protein loses function. For example, the gene may be modified to mutate an active site of protease, reverse transcriptase or integrase proteins. Rev-independent fragments of gag and env are described, for example, in WO01/46408 and U.S. Pat. Nos. 5,972,596 and 5,965,726. Typically, rev-independent HIV sequences that are modified to eliminate all enzymatic activities of the encoded proteins are used in the constructs of the invention. All the genes encoding gag, pol, env, tat, nef and vif can be made Rev-independent by altering the nucleotide sequence without affecting the protein sequence. The altered nucleotide compositions of the genes also reduce the probability of recombination with wildtype virus.

In the present invention, a “nucleic acid” molecule can include cDNA and genomic DNA sequences, RNA, and synthetic nucleic acid sequences. Thus, “nucleic acid” also encompasses embodiments in which analogs of DNA and RNA are employed.

### Protein Component

The combination immunization protocol of the invention for inducing an immune response includes a polypeptide component that comprises epitopes of the antigen of interest that stimulate a humoral and/or cellular immune response.

As used herein, the term “HIV polypeptide” or “HIV antigen” refers to any HIV peptide from any HIV strain or subtype and combinations thereof HIV polypeptides for use in the invention include gag, pol, env, vif, vpr, tat, rev, nef, and/or vpu; functional (e.g., immunogenic) fragments thereof, modified polypeptides thereof and combinations of these fragments and/or modified peptides. An HIV polypeptide for use in the invention can be from any of the various HIV strains and subtypes. Furthermore, an “HIV polypeptide” as defined herein is not limited to a polypeptide having the exact sequence of known HIV polypeptides, as there is considerable variation in sequences and new sequence are frequently identified.

Polypeptides, e.g., HIV polypeptides, used in the invention include proteins that have modifications to the native sequence, such as internal deletions, additions and substitutions, which are usually conservative in their nature.

“Wild-type” or “native” sequences, as used herein, refers to polypeptide encoding sequences that are essentially as they are found in nature, e.g., for HIV polypeptides, Gag and/or Env encoding sequences as found in other isolates such as Type C isolates (e.g., Botswana isolates AF110965, AF110967, AF110968 or AF110975 or South African isolates).

In some embodiments, e.g., where a pathogenic virus is the targeted disease, the protein component may be inactivated virus particles, e.g., aldrithiol-2 (AT-2)-inactivated particles, or may be virus-like particle (VLPs). Methods of inactivating particles are known in the art (see, e.g., Lifson, et al., AIDS Res Hum Retroviruses 20:772-787, 2004; Rossio, et al., J Virol 72:7992-8001, 1998). VLPs are non-replicating viral shells that contain the viral protein shell polypeptides and lack the viral polynucleotides required for normal viral replication. VLPs are generally composed of one or more viral proteins, such as, as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. (See, e.g., Schneider, et al., J Virol 7:4892-4903 (1997); Buonaguro., et al., J Virol 80:9134-9143 (2006); and Buonaguro, et al., Vaccine 25:5968-5977 (2007)).

In further embodiments, the protein component employed in an immunization protocol of the invention may be one or more recombinant polypeptide(s). Such polypeptides can be generated using methodology well known in the art.

In some embodiments, the proteins encoded by the nucleic acid component and/or included in the protein component may represent non-native sequences, including fragments, regions that are conserved, e.g., across strains of viruses, polypeptides representing consensus sequencers, or centalized or mosaic sequences with the aim to direct the immune response to specific regions of the virus or to address antigenic variability.

As used herein, the term “fragment” refers to a polypeptide having an amino acid sequence that is the same as part, but not all, of the amino acid sequence of the parent antigen from which it is derived or one of their functional equivalents. The fragments typically comprise at least one epitope. Accordingly, a fragment may comprise 6, 7, 8, 9, 10, 12, 14, 16, 18, or 20 or more consecutive amino acids from an antigen of interest.

An some embodiments the protein component for use in the invention comprises a cocktail of one or more individual peptides; or one or more peptides comprised by a polyepitopic peptide.

### Selection of Antigens

An antigen of interest may be an antigen from any disease for which it is desirable to induce a preventive and/or therapeutic immune response. Thus, the anteing of interest may be a tumor associated antigen, e.g., a melanoma antigen, or a breast, prostate, lung, colorectal, or renal antigen. Example of tumor-associated antigens include MAGE 1, 2, & 3; MART-1/Melan-A, gp100, carcinoembryonic antigen (CEA), HER-2, mucins (i.e., MUC-1), prostate-specific antigen (PSA), and prostatic acid phosphatase (PAP).

In some embodiments, the antigen of interest by be from an infection agent. Thus, the methods of the invention are useful in the prevention or treatment of diseases such as HIV, tuberculosis, malaria, influenza, hepatitis (e.g., HBV, HCV), CMV, herpes virus-induced diseases (e.g., HSV), Epstein Barr Virus (EBV), respiratory syncytial virus (RSV) and other viral infections, as well as diseases such as leprosy and non-malarial protozoan parasites such as toxoplasma. Accordingly, the antigen of interest may be from a virus such as a lentivirus, or another type of virus.

In some embodiments, the antigen of interest may be from a fungus or yeast, e.g., the causative agents of aspergillosis; thrush; cryptococcosis; and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans.

The methods of the invention may also be employed to prevent and/or treat bacterial infections. Accordingly, an antigen of interest may be from Helicobacter pyloris, Borelia burgdorferi, Legionella pneumophilia, Mycobacteria sps (such as. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, or Actinomyces israelli.

### Retroviral Antigens

Antigenic polypeptide sequences for provoking an immune response selective for a specific retroviral pathogen are known. In some embodiments of the invention, the vaccine regimen is administered to a patient with HIV-1 infection. With minor exceptions, the following discussion of HIV epitopes/immunogenic polypeptides is applicable to other retroviruses, e.g., SIV, except for the differences in sizes of the respective viral proteins. HIV antigens for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); the updated version of this data base is online and is incorporated herein by reference (http: followed by //hiv-web.lanl.gov/content/index)) and antigens derived from any of these isolates can be used in the methods of this invention. Immunogenic proteins can be derived from any of the various HIV isolates, including any of the various envelope proteins such as gp120, gp160 and gp41; gag antigens such as p24gag and p55gag, as well as proteins derived from pol, tat, vif, rev, nef, vpr, vpu.

### Co-Administration of Nucleic Acid and Protein Components

The nucleic acid and protein components employed in the co-immunization methods of the present invention are administered via co-immunization or simultaneous administration. Co-immunization or simultaneous administration can include administration as a co-mixture to the same body site location. Co-immunization can also include administration of either the nucleic acid component or protein component followed by administration within 48 hours of the previously non-administered component (for example, the nucleic acid, e.g., plasmid DNA, component is administered, followed within 48 hours by administration of the protein component; or the protein component may be administered, followed within 48 hours by administration of the nucleic acid component). In some embodiments, administration of the two components is performed within 24 hours of one another. In some embodiments, administration of the two components is performed within 8 hours of one another. In some embodiments, administration of the two components is performed within 4 hours of one another. In some embodiments, administration of the two components is performed within 1 hour of one another. In some embodiments, administration of the two components is performed within 30 minutes of one another. In some embodiments, administration of the two components is performed within 10 minutes of one another, e.g., within 1 to 5 minutes of one another. In typical embodiments, separate administration is performed to the same body site location, e.g., to the upper arm, to the thigh, to the torso, to the buttocks, etc. In other embodiments, the combination nucleic acid protein vaccine components can be administered to multiple body sites, either together or separately.

In some embodiments, the nucleic acid and protein components of the invention are co-administered to an individual with the proviso that the individual is immunologically naïve and has not previously been the subject of an administration of either a nucleic acid encoding the antigen of interest, e.g., HIV env, or a protein vaccine comprising the antigen of interest, e.g., comprising an HIV env protein.

The components of the immunization protocols of the invention may be administered to individuals who do not have a disease, e.g., immunologically naïve individuals who have, not been infected with the organism for which it is desired to elicit an immunological response. Thus, e.g., a vaccine regimen of the invention can be used for prevention of a disease, e.g., infection with an agent such as a viral agent. For example, an HIV vaccine comprising a nucleic acid and protein component administered as described herein may be administered to individuals at risk for HIV infection.

In some embodiments, the vaccine components may be administered to an individual who has a disease, e.g., has cancer or is infected with a pathogenic organism. Thus, in some embodiments, the vaccine is administered to an individual who already is infected with a bacteria, virus, fungus, parasite, or the like.

In some embodiments, an immunization regimen of the invention targets a retrovirus, e.g., HIV. Accordingly, in some embodiments, HIV vaccines may be administered to individuals who may be at risk for HIV infection, e.g., individuals who are in high risk groups such as individuals who are exposed to HIV. In some embodiments, the vaccine regimen of the invention may be administered therapeutcially to an HIV infected individual, typically an HIV-1-infected human. Typically, such individuals are undergoing or have undergone ART therapy. Thus, the compositions can be used in combination with common anti-retroviral therapeutics including reverse transcriptase inhibitors and protease inhibitors. Such inhibitors are well known in the art. Examples of reverse transcriptase inhibitors include nucleoside analogs, e.g., AZT and other anti-retroviral nucleoside analogs, and nonnucleoside reverse transcriptase inhibitors (NNRTIs) such as Delavirdine and Nevirapine. A detailed review can be found in “Nonnucleoside Reverse Transcriptase Inhibitors” AIDS Clinical Care (10/97) Vol. 9, No. 10, p. 75. Protease inhibitors include: SAQUINAVIR (Invirase); INDINAVIR (Crixivan); and RITONAVIR (Norvir).

Additional classes of antiretroviral drugs for clinical use include inhibitors of retrovirus entry and integrase inhibitors. Such drugs can also be used in combination with the immunogenic compositions described herein.

### Administration and Pharmaceutical Formulations

The nucleic acid component and protein components administered in accordance with the invention are co-administered to a mammalian host. The mammalian host usually is a human or a primate. In some embodiments, the mammalian host can be a domestic animal, for example, canine, feline, lagomorpha, rodentia, rattus, hamster, murine. In other embodiment, the mammalian host is an agricultural animal, for example, bovine, ovine, porcine, equine, etc.

### Administration of Nucleic Acid Component

In the methods of the invention, the nucleic acid component is often directly introduced into the cells of the individual receiving the immunogenic composition. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and WO 98/04720. Examples of DNA-based delivery technologies include, “naked DNA”, facilitated (bupivicaine, polymers, peptide-mediated) delivery, and cationic lipid complexes or liposomes. The nucleic acids can be administered using ballistic delivery as described, for instance, in U.S. Pat. No. 5,204,253 or pressure (see, e.g., U.S. Pat. No. 5,922,687). Using this technique, particles comprised solely of DNA are administered, or in an alternative embodiment, the DNA can be adhered to particles, such as gold particles, for administration.

In some embodiments, e.g., where a nucleic acid component of the invention is encoded by a viral vector, the nucleic acid component can be delivered by infecting the cells with the virus containing the vector. This can be performed using any delivery technology, e.g., as described in the previous paragraph.

In some embodiments, the immunogenic compositions of the invention are administered by injection or electroporation, or a combination of injection and electroporation.

Therapeutic quantities of nucleic acids, e.g., plasmid DNA, can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins. If required, supercoiled DNA can be isolated from the open circular and linear forms using centrifugation, gel electrophoresis or other methods.

Purified plasmid DNA can be prepared for injection using a variety of formulations. The simplest of these is reconstitution of lyophilized DNA in sterile phosphate-buffer saline (PBS). This approach, i.e., “naked DNA,” is particularly suitable for intramuscular (IM) or intradermal (ID) administration. Alternatively, other physiologically compatible buffer formulations or sterile water can be used for DNA administration.

Therapeutic quantitites of viral vectors, e.g., poxvirus vectors, adenovirus vectors, etc. can also be obtained using known methodology.

The nucleic acids to be administered to an individual in accordance with the methods of the invention are formulated for pharmaceutical administration. While any suitable carrier known to those of ordinary skill in the art may be employed in the pharmaceutical compositions of this invention, the type of carrier will vary depending on the mode of administration. For parenteral administration, including intranasal, intradermal, subcutaneous or intramuscular injection or electroporation, the carrier preferably comprises water, saline, and optionally an alcohol, a fat, a polymer, a wax, one or more stabilizing amino acids or a buffer. General formulation technologies are known to those of skill in the art (see, for example, Remington: The Science and Practice of Pharmacy (20th edition), Gennaro, ed., 2000, Lippincott Williams & Wilkins; Injectable Dispersed Systems: Formulation, Processing And Performance, Burgess, ed., 2005, CRC Press; and Pharmaceutical Formulation Development of Peptides and Proteins, Frkjr et al., eds., 2000, Taylor & Francis).

Nucleic acids can be administered in solution (e.g., a phosphate-buffered saline solution) by injection, usually by an intra-arterial, intravenous, subcutaneous or intramuscular route. Suitable quantities of nucleic acids, e.g., plasmid or naked DNA, or RNA, can be about 1 μg to about 10 mg, preferably 0.1 to 10 mg, but lower levels such as 1-10 μg can be employed. In general, the dose of nucleic acid composition is from about 10 μg to 50 mg for a typical 70 kilogram patient. Subcutaneous or intramuscular doses for naked nucleic acid (typically DNA encoding a fusion protein) will range from 0.01 mg to 20 mg for a 70 kg patient in generally good health. Dosages are sufficient to stimulate an immune response. In some embodiments, the dose of nucleic acid is about 0.02, 0.05, 0.1, 0.2, 0.5 mg/kg body weight. For example, an HIV DNA vaccine, e.g., naked DNA or polynucleotide in an aqueous carrier, can be injected into tissue, e.g., intramuscularly or intradermally, in amounts of from 10 μl per site to about 1 ml per site. The concentration of polynucleotide in the formulation is usually from about 0.1 μg/ml to about 10 mg/ml.

Nucleic acid components of the immunogenic compositions can be administered once or multiple times. However, at least the first administration is performed with co-delivery of the protein component of the immunogenic composition. DNA vaccination is performed more than once, for example, 2, 3, 4, 5, 6, 7, 8, or 10 or more times as needed to induce the desired response (e.g., specific antigenic response or proliferation of immune cells) or to maintain the immune response by periodic vaccination, for example, once per year. Multiple administrations can be administered, for example, monthly, or more or less often, as needed, for a time period sufficient to achieve the desired response.

Nucleic acid components are administered by methods well known in the art as described in Donnelly et al. (Ann. Rev. Immunol. 15:617-648 (1997)); Felgner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), each of which is incorporated herein by reference. One skilled in the art would know that the choice of a pharmaceutically acceptable carrier, including a physiologically acceptable compound, depends, for example, on the route of administration of the expression vector.

In some embodiments, the nucleic acid vectors are administered by liposome-based methods, electroporation or biolistic particle acceleration. A delivery apparatus (e.g., a “gene gun”) for delivering DNA into cells in vivo can be used. Such an apparatus is commercially available (e.g., BioRad, Hercules, Calif., Chiron Vaccines, Emeryville, Calif.). Naked DNA can also be introduced into cells by complexing the DNA to a cation, such as polylysine, which is coupled to a ligand for a cell-surface receptor (see, for example, Wu, G. and Wu, C. H. (1988) J. Biol. Chem. 263:14621; Wilson et al. (1992) J. Biol. Chem. 267:963-967; and U.S. Pat. Nos. 5,166,320; 6,846,809; 6,733,777; 6,720,001; 6,290,987). Liposome formulations for delivery of naked DNA to mammalian host cells are commercially available from, for example, Encapsula NanoSciences, Nashville, Tenn. An electroporation apparatus for use in delivery of naked DNA to mammalian host cells is commercially available from, for example, Inovio Biomedical Corporation, San Diego, Calif.

Expression vectors, RNA molecules (that encode the antigen of interest) and the like can be delivered to the interstitial spaces of tissues of a person (see, e.g., Felgner et al., U.S. Pat. Nos. 5,580,859, and 5,703,055). Administration of the nucleic acid component to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

### Administration of Protein Component

The protein component of an immunogenic composition of the invention can be formulated using methodology well know to those skilled in the pharmaceutical art. Such compositions can be administered in dosages and by techniques well known to those skilled in the medical arts taking into consideration such factors as the age, sex, weight, and condition of the particular patient, and the route of administration.

Typical dosages can range from about 0.01 mg/kg body weight up to and including about 0.5 mg/kg body weight. In some embodiments, the dose of polypeptide is about 0.01, 0.02, 0.05, 0.08, 0.1, 0.2, 0.3, 0.4, 0.5 mg/kg body weight. Dosages are sufficient to stimulate an immune response.

The protein can be administered by any route, for example, including without limitation, enterally (i.e., orally) or parenterally, e.g., intravenously, intramuscularly, subcutaneously, intradermally, intranasally, or inhalationally.

Protein components of the immunogenic compositions can be administered once or multiple times. However, at least the first administration is performed with co-delivery of the nucleic acid component of the immunogenic composition. Protein vaccination can be performed more than once, for example, 2, 3, 4, 5, 6, 7, 8, or 10 or more times as needed to induce the desired response (e.g., specific antigenic response or proliferation of immune cells) or to maintain the immune response by periodic vaccination, for example, once per year. Multiple administrations can be administered, for example, monthly, or more or less often, as needed, for a time period sufficient to achieve the desired response.

Formulations and Administration with Other Agents

The vaccine compositions, nucleic acid or protein, can include various excipients, adjuvants, carriers, auxiliary substances, modulating agents, and the like.

The immunogenic compositions are co-administered to a patient in an amount sufficient to elicit a therapeutic effect, e.g., a CD8⁺, CD4⁺, and/or antibody response to the antigen of interest to which the nucleic acid/protein components are directed. This can be an amount that at least partially arrests or slows symptoms and/or complications of a disease, e.g., HIV infection. An amount adequate to accomplish this is defined as “therapeutically effective dose.” Amounts effective for this use will depend on, e.g., the particular composition of the regimen administered, the manner of administration, the stage and severity of the disease, the general state of health of the patient, and the judgment of the prescribing physician.

The compositions may be delivered in a physiologically compatible solution such as sterile PBS in a volume of, e.g., one ml. The component (either protein or nucleic acid component, or both) may also be lyophilized prior to delivery. As well known to those in the art, the dose may be proportional to weight.

The nucleic acid/protein co-immunization compositions can be administered alone, or can be co-administered or sequentially administered with other immunological, antigenic, vaccine, or therapeutic compositions.

Compositions that may also be administered with the immunogenic nucleic acid and protein components include other agents to potentiate or broaden the immune response, e.g., IL-2 or CD40 ligand, which can be administered at specified intervals of time, or continuously administered. For example, IL-2 can be administered in a broad range, e.g., from 10,000 to 1,000,000 or more units. Administration can occur continuously following vaccination.

In some embodiments, the methods of the invention comprise administering a molecule adjuvant such as IL-15, IL-12, or IL-2. Other adjuvants that can be used with the vaccines of the present invention include lectins, growth factors, cytokines and lymphokines such as alpha-interferon, gamma interferon, platelet derived growth factor (PDGF), granulocyte-colony stimulating factor (GCSF), granulocyte macrophage colony stimulating factor (GM-CSF), tumor necrosis factor (TNF), epidermal growth factor (EGF), IL-1, IL-4, IL-6, IL-8, and IL-10, as well as nucleic acids encoding these agents.

In some embodiments, the method of the invention comprise administering traditional adjuvants. Such adjuvants are well known to those of skill in the art. Adjuvants suitable for co-administration with the vaccines of present invention should be ones that are potentially safe, well-tolerated and effective in people. Examples of adjuvants include but are not limted to QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59. (See, e.g., Kim et al., Vaccine 18:597 (2000) and references therein).

The nucleic acid and/or polypeptide components can additionally be complexed with other components such as peptides, polypeptides, lipids, and carbohydrates for delivery. For example, expression vectors, i.e., nucleic acid vectors that are not contained within a viral particle, can be complexed to particles or beads that can be administered to an individual, for example, using a gene gun.

As explained above, the nucleic acid and protein components can be delivered via a variety of routes. Typical delivery routes include parenteral administration, e.g., intradermal, intramuscular or subcutaneous routes. Other routes include oral administration, intranasal, and intravaginal routes. In such compositions the nucleic acid and/or protein can be in admixture with a suitable carrier, diluent, or excipient such as sterile water, physiological saline, glucose or the like.

The nucleic acid and/or protein component can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. Nos. 5,846,978, 5,663,169, 5,578,597, 5,502,060, 5,476,874, 5,413,999, 5,308,854, 5,192,668, and 5,187,074.

The nucleic acid and/or protein components can be incorporated, if desired, into liposomes, microspheres or other polymer matrices (see, e.g., Felgner et al., U.S. Pat. No. 5,703,055; Gregoriadis, Liposome Technology, Vols. I to III (2nd ed. 1993). Liposomes, for example, which consist of phospholipids or other lipids, are nontoxic, physiologically acceptable and metabolizable carriers that are relatively simple to make and administer. Liposomes include emulsions, foams, micelles, insoluble monolayers, liquid crystals, phospholipid dispersions, lamellar layers and the like.

Liposome carriers can serve to target a particular tissue or infected cells, as well as increase the half-life of the vaccine. In these preparations, the protein component and/or nucleic acid component can be formulated to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired immunogen of the invention can be directed to the site of lymphoid cells, where the liposomes then deliver the immunogen(s).

Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

### Assessment of Immunogenic Response

To assess an individual's immune system during and after vaccination and to further evaluate the vaccination regimen, various parameters can be measured. Measurements to evaluate vaccine response include but are not limited to: antibody measurements in the plasma, serum, saliva, or other body fluids; analysis of in vitro cell proliferation in response to a specific antigen, indicating the function of CD4′ cells; analysis of cytokine production of lymphocytes after stimulation with the specific antigen or with pools of peptides of the specific antigen; and analysis of neutralizing activity found in the serum or plasma using virus inhibition assays well known in the art. Such assays are well known in the art.

Other measurements of immune response include assessing CD8+ responses. These techniques are well known. CD8+ T-cell responses can be measured, for example, by using tetramer staining of fresh or cultured PBMC (see, e.g., Altman, et al., Proc. Natl. Acad. Sci. USA 90:10330, 1993; Altman, et al., Science 274:94, 1996), or γ-interferon release assays such as ELISPOT assays (see, e.g., Lalvani, et al., J. Exp. Med. 186:859, 1997; Dunbar, et al., Curr. Biol. 8:413, 1998; Murali-Krishna, et al., Immunity 8:177, 1998), or by using functional cytotoxicity assays.

In other embodiments, the antibody response is measured (see, e.g., the Examples section for methodology). The combination nucleic acid/protein vaccines of the invention typically provide improved immunological memory and longer lasting antibody response in comparison to standard prime boost protocols where the nucleic acid and protein components are administered sequentially with a long time frame (e.g., 2 weeks or more) separating administration of a nucleic acid component from a protein component.

### Viral Titer

In therapeutically vaccinated, HIV infected humans, viremia can also be determined as a measure of therapeutic efficacy, e.g., for an immunogenic composition comprising HIV antigens and nucleic acids encoding such antigens. Viremia is measured by assessing viral RNA copies in a patient. There are a variety of methods of perform this. For example, plasma HIV RNA concentrations can be quantified by either target amplification methods (e.g., quantitative RT polymerase chain reaction [RT-PCR], Amplicor HIV Monitor assay, Roche Molecular Systems; or nucleic acid sequence-based amplification, [NASBA®], NucliSens™ HIV-1 QT assay, Organon Teknika) or signal amplification methods (e.g., branched DNA [bDNA], Quantiplex™ HIV RNA bDNA assay, Chiron Diagnostics). The bDNA signal amplification method amplifies the signal obtained from a captured HIV RNA target by using sequential oligonucleotide hybridization steps, whereas the RT-PCR and NASBA® assays use enzymatic methods to amplify the target HIV RNA into measurable amounts of nucleic acid product. Target HIV RNA sequences are quantitated by comparison with internal or external reference standards, depending upon the assay used.

### Kits

The invention also provides kits comprising the nucleic acid and proteins components to be administered in accordance with the methods described herein. Such a kit can comprise for example, a container that includes one or more of the nucleic acid vectors contained in a vessel and a separate vessel containing the protein form of the antigen. Thus, a kit of the invention can comprise a protein form of the antigen separate from the nucleic acid form of the antigen, and a nucleic acid component where the nucleic acid component can comprise individual vectors contained in separate vessels.

The kit may also include other components, e.g., for mixing with one or both of the compositions before administration, such as diluents, carriers, adjuvants, and the like.

### EXAMPLES

### Example 1

### DNA/Protein Combination Vaccines by DNA and Protein Co-Immunization at the Same Site

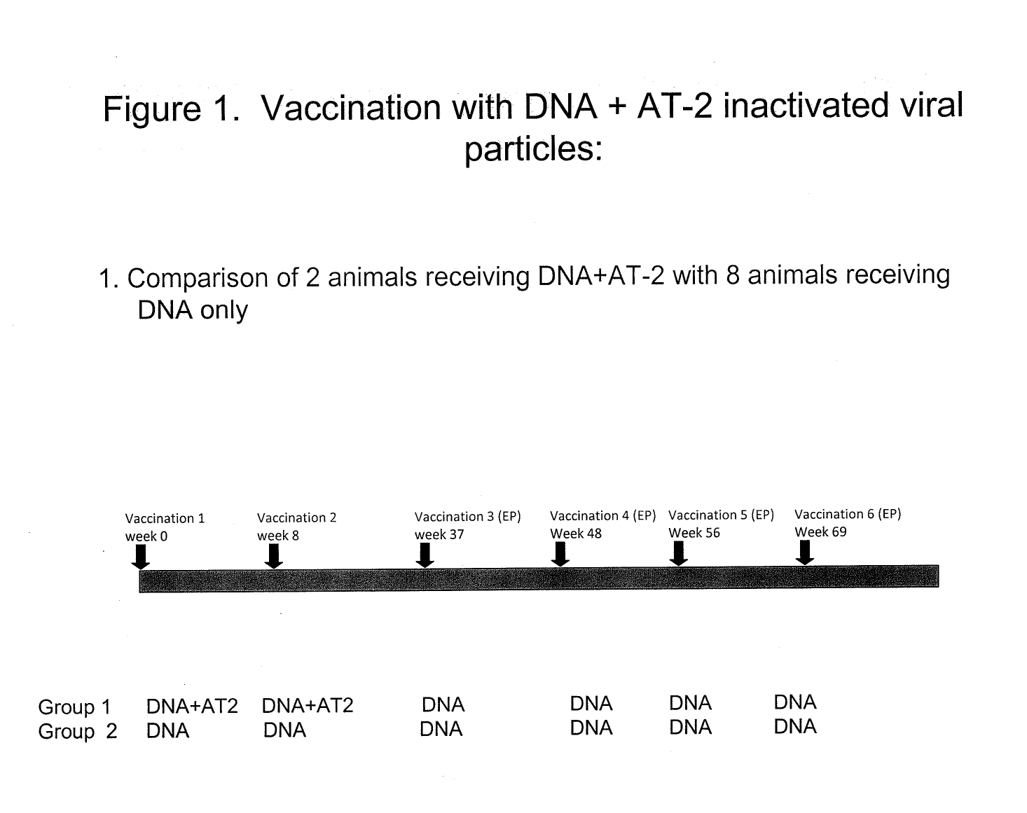

In this example, whole inactivated virus particles were used as the source of protein. These particles contain all viral proteins, including envelope, gag, and pol. Different groups of Indian macaques were vaccinated with DNA alone (n=8), or co-immunized with DNA+protein at the same site (n=2). The two animals that were vaccinated with DNA+protein received the protein component in the form of AT-2 whole inactivated SIV viral particles (Lifson, et al., AIDS Res Hum Retroviruses 20:772-787, 2004; Rossio, et al., J Virol 72:7992-8001, 1998). We then compared the two animals receiving DNA+AT-2 particles with the eight animals receiving DNA only. Both DNA and protein (AT-2 inactivated viral particles) were administered to macaques by needle and syringe for the first 2 vaccinations. Vaccination was by intramuscular (IM) in the same site with no adjuvants. The animals were rested for 7 months and then boosted four times with DNA only. Both groups received the same DNA mixtures administered by electroporation (EP). The times of administration of the vaccines are shown in FIG. 1.

The following group of DNAs were mixed and delivered IM; in addition to the plasmids expressing SIV antigens, a plasmid expressing macaque IL-12 DNA was included in the mix. For the first 2 vaccinations, the mix was adjusted to a total of 3 mg/each antigen type and included:

Gag:
2S CATEDX, 1.5 mg
21S MCP3p39, 1.5 mg

Env:
72S CATEenv, 1.5 mg
73S MCP3-Env, 1.5 mg

Pol, Nef Tat Vif:
44S CATE-PolNTV, 1.5 mg
155S CATE-PolNT, 1.5 mg

IL-12
AG3, 3 mg.

These plasmids have been well described (Rosati, et al., PNAS, 106:15831-15836 (2009)).

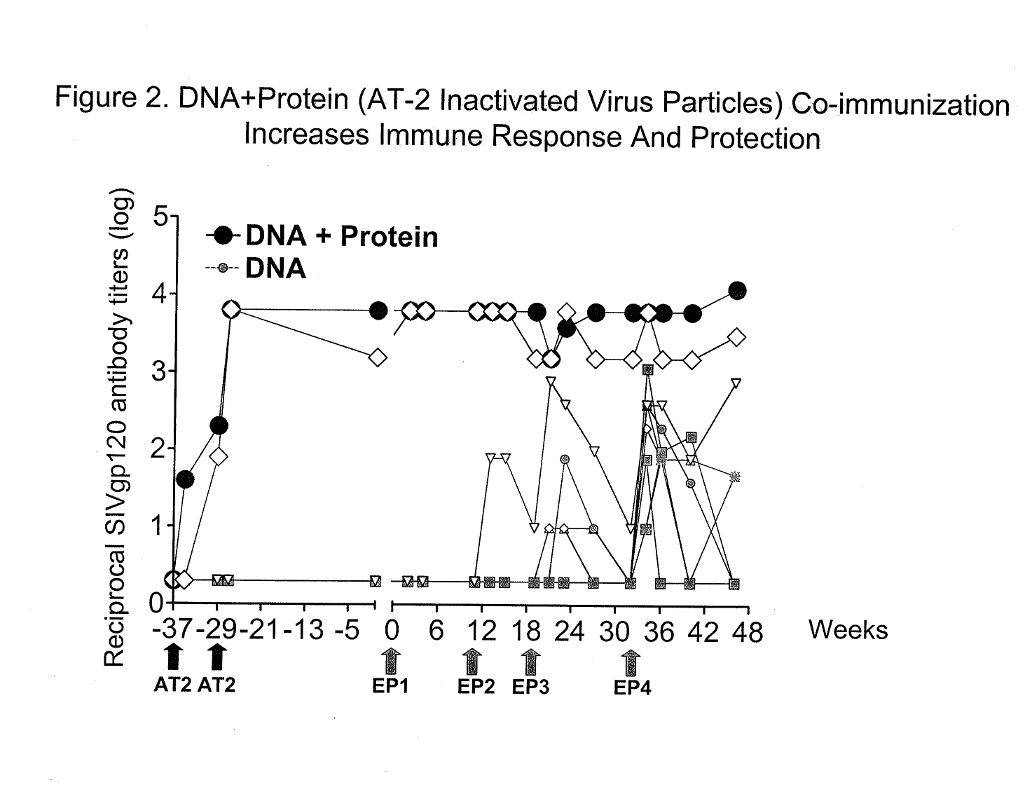

The results showed that co-immunization with DNA and AT-2-inactivated virus particles increased the immune responses. Animals co-immunized with DNA+protein developed high Ab responses from the start (FIG. 2), and also had high cellular immune responses.

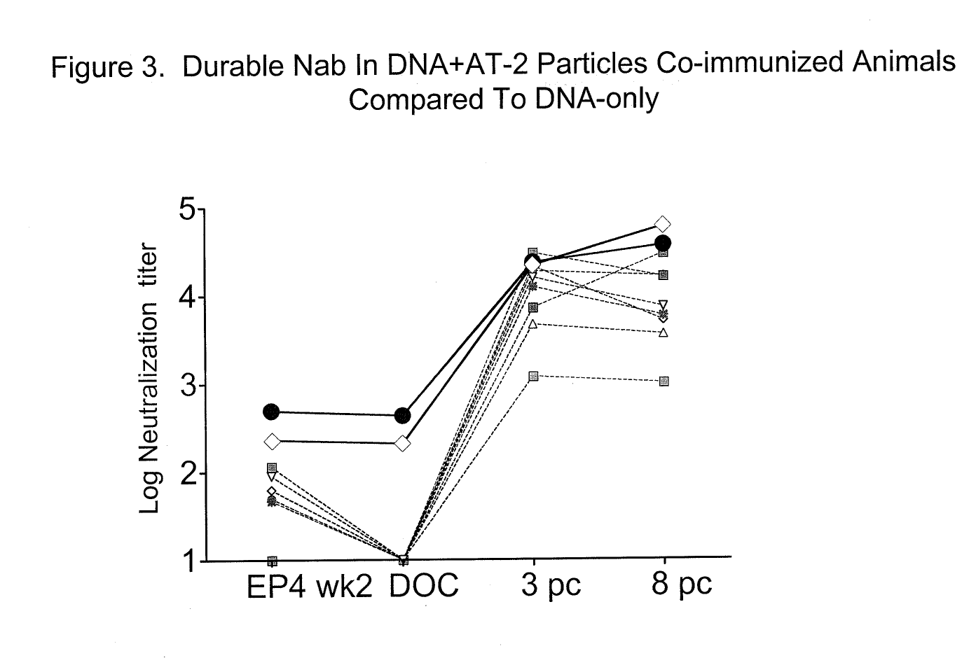

Higher levels of neutralizing antibodies were also detected in DNA+protein (AT-2 Particles) co-immunized animals compared to DNA-only vaccinated animals (FIG. 3). These neutralizing antibodies were more durable over time in the DNA+protein co-immunized animals.

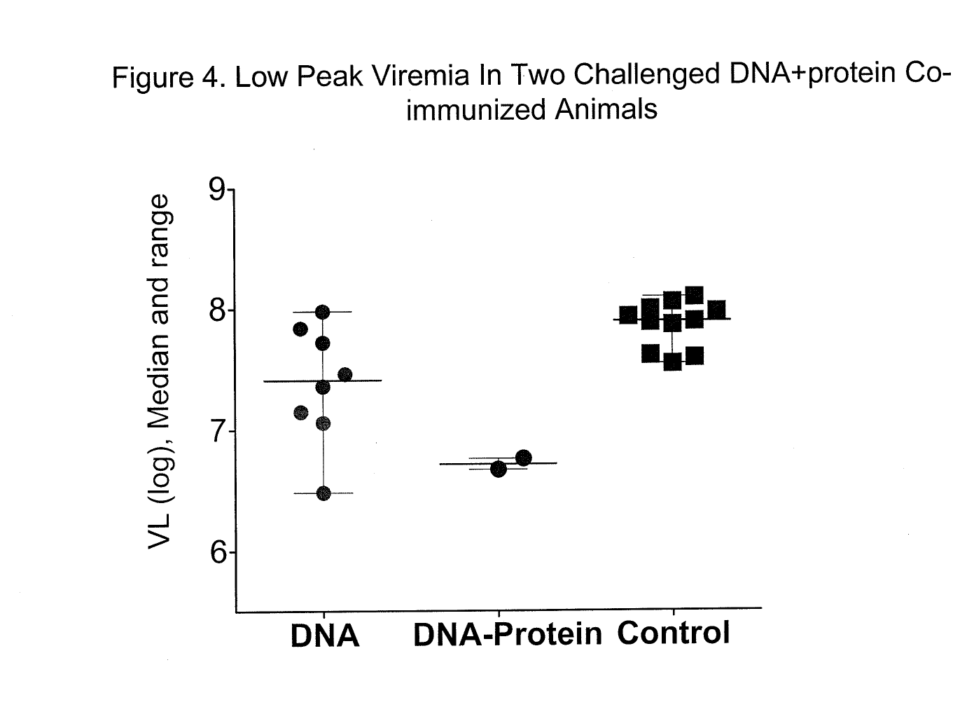

When animals were challenged with SIVmac251, those receiving the DNA/AT-2 particle combination vaccine exhibited low peak viremia (FIG. 4).

### Example 2

### DNA/Protein Combination Vaccines by DNA Electroporation and Protein Co-Immunization at the Same Site

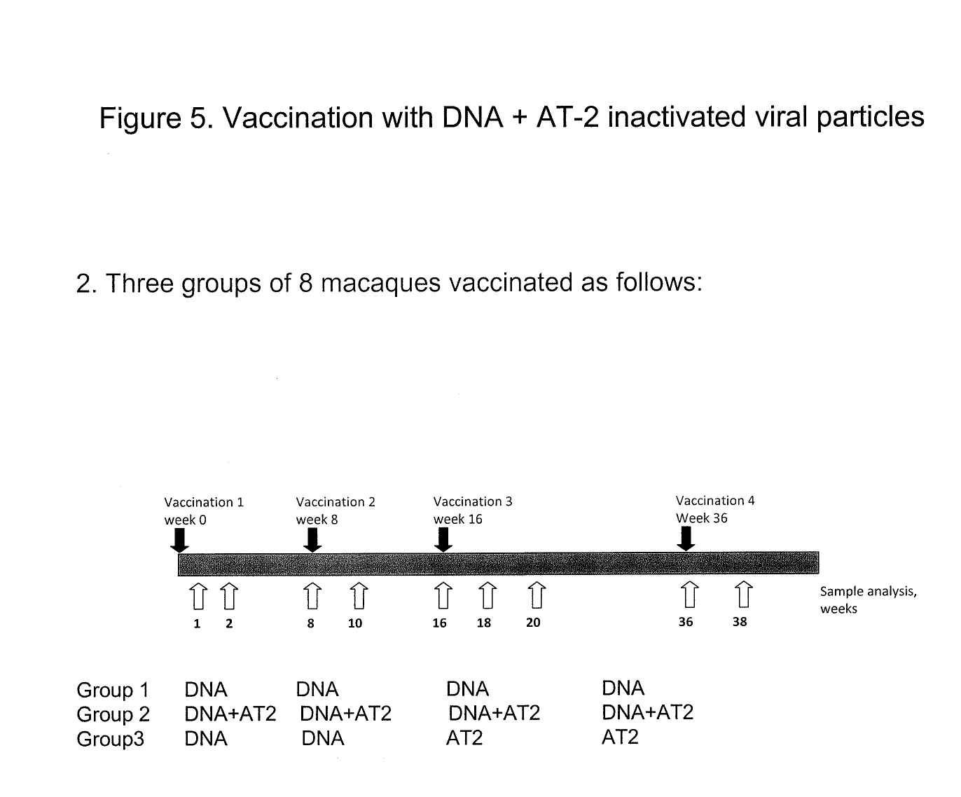

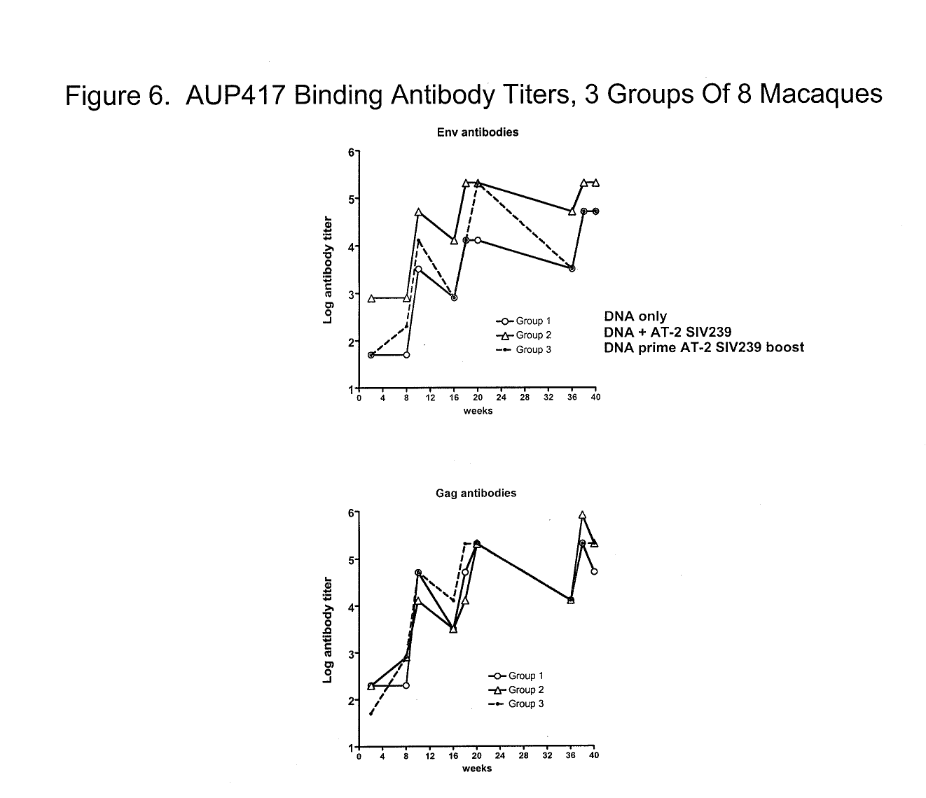

In this example, three groups of eight macaques were vaccinated with either DNA (Group1) or with DNA and AT-2 inactivated VP (Group2) four times (FIG. 5). The results also showed that co-delivery of DNA and AT-2 protein particles increased humoral immune responses to env in macaques, but did not alter significantly the Gag Ab responses. FIG. 6 shows the average Ab responses. Three groups of eight macaques were vaccinated 4× at weeks 0, 8, 16 and 36. The average antibody responses to the Env and Gag proteins of SIVmac are shown. The groups are: Group 1, DNA only. Group 2, DNA+protein (AT-2 particles) co-delivery in the same site at the same time. Group 3, DNA (weeks 0 and 8) and protein (AT-2) (weeks 16 and 36) immunizations sequentially. DNA delivery was intramuscular followed by electroporation.

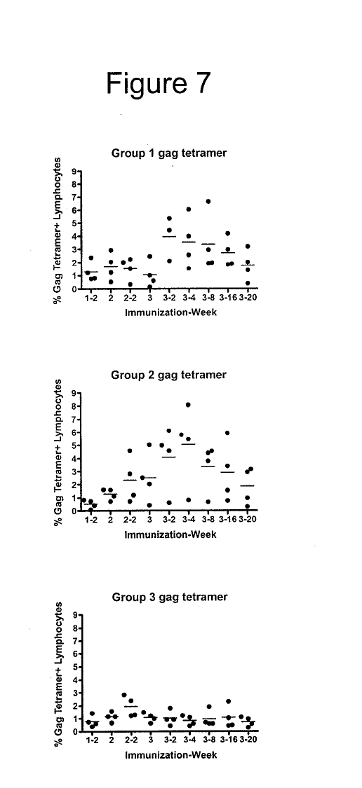

FIG. 7 shows a comparison of co-immunization of DNA+protein (AT-2 particles) versus DNA vaccination alone in inducing cellular immune responses in the lung, performed by analyzing lymphocytes recovered from the lung after bronchioalveolar lavage (BAL). Cellular immune responses in BAL of four animals (Mamu-A*01 positive haplotype) per group were determined by Gag tetramer staining Group 1: DNA only; Group 2: DNA+protein co-immunized; Group 3: 2×DNA (vaccination 1 and 2) followed by protein only boost (vaccination 3). Analysis of the gag responses show that DNA boosts the immune response every time (Groups 1 and 2), whereas the protein alone (Group 3) did not boost the cellular immune response after the 3rd vaccination.

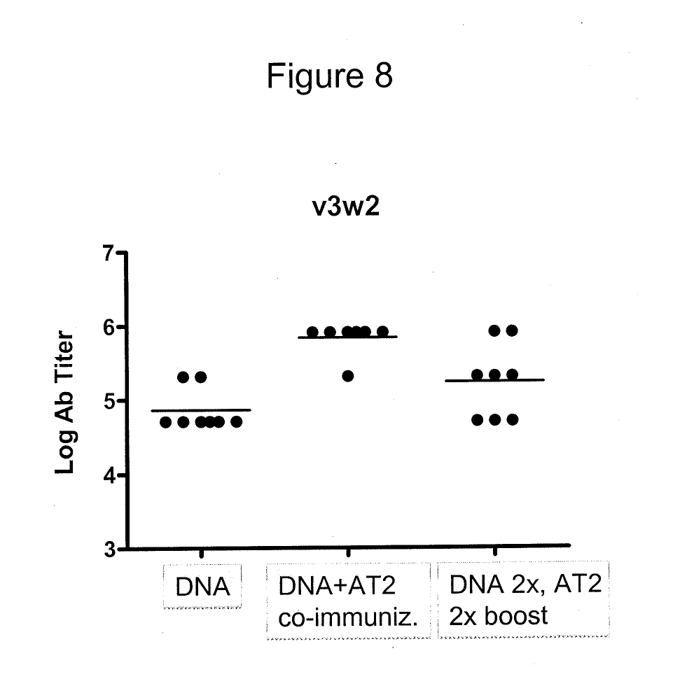

The Env Antibody response obtained after DNA and AT-2 particle co-immunization was enhanced, as determined by detailed analysis of the immune response in each animal over time. FIG. 8 shows the result of end-point dilution titers determined by Elisa for all animals in the three groups at 2 weeks after the third vaccination (week 18, see FIG. 5). The results of Group 2 were superior to Group 1 (DNA immunization) (p=0.0002, Kruskal-Wallis).

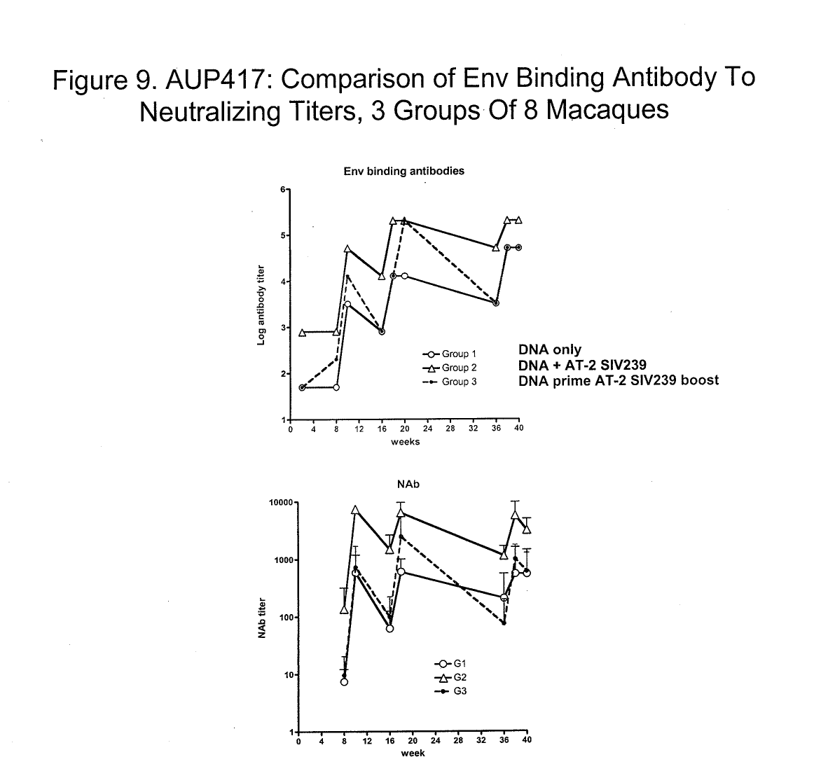

FIG. 9 is a comparison of binding Ab levels for env (top, same as FIG. 6) to Neutralizing Ab (Nab) titers to lab-adapted SIVmac251 (bottom). Group 2 developed maximal Nab titers after 2 vaccinations. The other groups developed lower and less durable Nab.

### Example 3

### DNA/Protein Co-Immunization Using HIV gp120 Env (HIV Isolate BaL)

An additional vaccine experiment in macaques was performed using a different antigen, HIV gp120 Env (HIV isolate BaL). This protein preparation was injected in the same site and immediately after the DNA at weeks 0 and 4. Blood was analyzed for anti-Env and anti-Gag antibodies and also for HIV Neutralizing Antibodies at week 6.

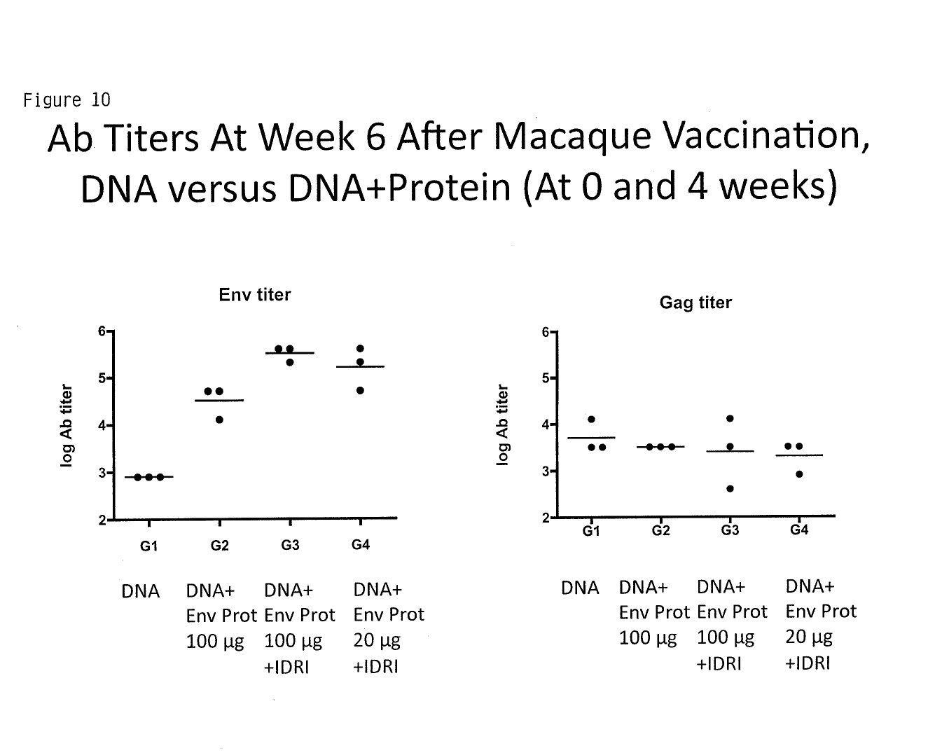

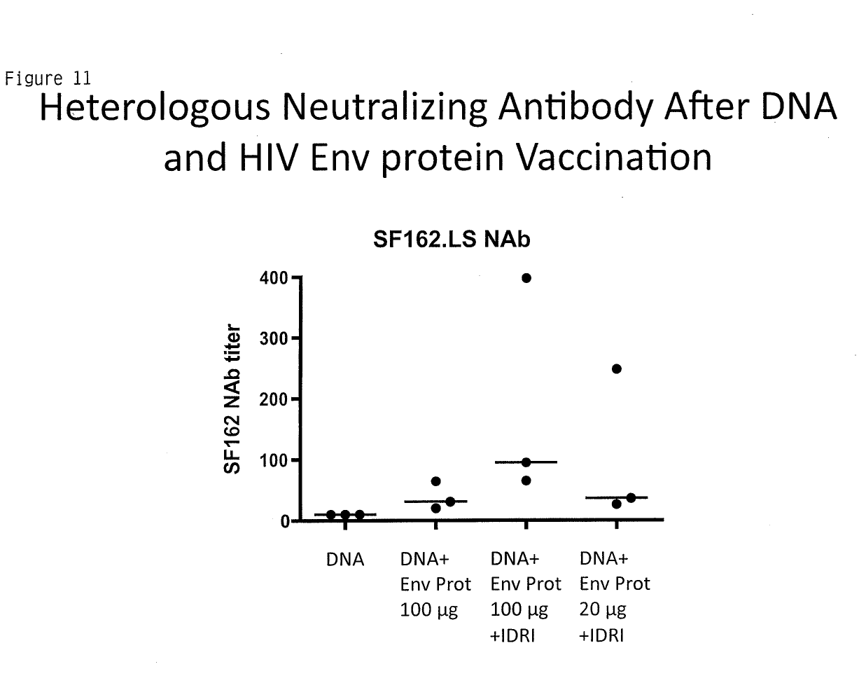

FIG. 10 shows that the animals co-immunized with DNA and different Env protein formulations had higher antibody titers. In addition to binding Abs, this vaccination also allowed the development of heterologous neutralizing Abs, as shown in FIG. 11. Both un-adjuvanted Env protein and Env mixed with IDRI EM005 adjuvant increased the Env Ab titers.

For Example 3, four groups of three macaques were vaccinated at 0 and 4 weeks as follows:

### Group 1 DNA

Group 2: DNA and protein (purified HIV gp120BaL, 100 μg in Phosphate Buffered Saline)
Group 3: DNA and protein (purified HIV gp120BaL, 100 μg mixed with 100 μg IDRI EM005 adjuvant)

Group 4: DNA and protein (purified HIV gp120BaL, 20 μg mixed with 100 μg IDRI EM005 adjuvant).

All groups received the same amount and formulation of DNA antigens, as shown in the following Table. DNA was formulated in PBS.

| | Amount: |
|---|---|
| DNA vectors | μg/animal |
| 206S: (p55gag SIVmac239) | 250 |
| 209S: (MCP3-p39gag SIVmac239) | 250 |
| 217H (HIV Env Bal gp160) | 500 |
| AG157 (rhesus mac. IL-12) | 100 |

The results from the experiments performed above demonstrated that, surprisingly, co-vaccination with DNA and protein increased the magnitude and longevity of immune responses. This indicates that this combination led to superior results in terms of memory induction, generating more B and T cells with superior immunological memory characteristics against the desired antigens compared to traditional sequence administration of DNA and protein.

These properties of a vaccine, e.g., an HIV vaccine, are highly desirable, because they lead to faster development of superior immune responses compared to DNA only, protein only, or sequential administration of DNA and protein, as it is traditionally done in prime-boost combinations.

### Methods

### IM Injection of DNA by Needle and Syringe in 2 Macaques of Example 1.

The following group of DNAs were mixed and delivered IM; in addition, a plasmid expressing macaque IL-12 DNA was included in the mix: The mix was adjusted to a total of 3 mg/each antigen type and included:

Gag:
2S CATEDX, 1.5 mg
21S MCP3p39, 1.5 mg

Env:
72S CATEenv, 1.5 mg
73S MCP3-Env, 1.5 mg
Pol, Nef Tat Vif
44S CATE-PolNTV, 1.5 mg
155S CATE-PolNT, 1.5 mg

IL-12:
AG3 WLVrhIL12opt, 3 mg

The animals were also injected at the same site with 250 μl of AT-2 inactivated SIVmac239 viral particles containing the equivalent of 43 μg of p25gag. This material was injected half intramuscularly in the same site, and half intradermally above the muscle.

### IM Injection of DNA Followed by Electroporation in the Macaques of Example 2.

The following DNAs were injected IM in 8 macaques followed by in vivo electroporation using the Inovio Elgen device as specified by the manufacturer.

| | |
|---|---|
| 206S gag | 250 μg |
| 209S MCP3gag p39 | 250 μg |
| 99S Env239 | 500 μg |
| 216S MCP3-pol | 250 μg |
| 103S LAMP-pol | 250 μg |
| 147S LAMP-NTV | 500 μg |
| AG157 rmIL-12 | 500 μg |

At the same time, AT-2 inactivated purified SIVmac239 particles were injected in the same site (half IM and half ID in the same site). The volume of AT-2 particle solution was 250-400 μl and contained the equivalent of 43 μg p27gag.

The results of the simultaneous vaccination by DNA and protein as AT-2 inactivated viral particles were superior and surprising, compared to either DNA alone or protein alone vaccination. DNA alone produced both cellular and humoral immune response (see, e.g., Rosati, et al., PNAS, 106:15831-15836 (2009)), but the humoral immune response was lower and had inferior longevity compared to DNA+AT-2 particles. Protein only inoculation in the form of AT-2 inactivated viral particles, boosted antibody production but did not boost cellular immune responses.

The singular terms “a,” “an,” and “the” include plural referents unless context clearly indicates otherwise. The term “plurality” refers to two or more. It is further to be understood that all base sizes or amino acid sizes, and all molecular weight or molecular mass values, given for nucleic acids or polypeptides are approximate, and are provided for description.

The above examples are provided to illustrate the invention but not to limit its scope. Other variants of the invention will be readily apparent to one of ordinary skill in the art and are encompassed by the appended claims.

All publications, patents, accession numbers, and patent applications cited in this specification are hereby incorporated herein by reference in their entirety for their disclosures of the subject matter in whose connection they are cited herein.

What is claimed is:

1. A method of generating a preventive or therapeutic immune response in an individual, the method comprising co-administering two immunization components to generate an immune response: wherein one component is a nucleic acid component that encodes an antigen and (ii) a second component is a protein component that comprises the antigen of interest,
wherein the two components are administered to the individual at the same site and wherein administration of the two component at the same time enhances the immune response compared to administration of either component alone or sequentially.

2. The method of claim 1, wherein administering the nucleic acid component comprises administering at least two expression vectors encoding the antigen of interest, wherein the two expression vectors are formulated for administration separately or are formulated for administration together.

3. The method of claim 1, wherein the individual has cancer.

4. The method of claim 1, wherein the individual is immunologically naïve with respect to the antigen of interest.

5. The method of claim 1, wherein the individual is infected with a pathogenic agent.

6. The method of claim 5, wherein the pathogenic agent is a virus.

7. The method of claim 6, wherein the virus is a retrovirus.

8. The method of claim 1, wherein the nucleic acid component comprises one or more plasmid vectors that encode the antigen of interest.

9. The method of claim 1, wherein the nucleic acid component comprises one or more viral vectors that encode the antigen of interest.

10. The method of claim 1, wherein the site of administration is muscle.

11. The method of claim 1, wherein the antigen of interest is an HIV antigen.

12. The method of claim 1, wherein the protein component comprises inactivated viral particles.

13. The method of claim 11, wherein the HIV antigen is an envelope protein antigen.

14. The method of claim 13, wherein the protein component comprises recombinant envelope protein.

15. The method of claim 14, wherein the envelope protein is gp120 or as 140 trimer.

16. The method of claim 1, wherein the nucleic acid component and protein component are administered with an adjuvant.

17. The method of claim 1, wherein the immunogenic composition comprises at least one additional nucleic acid component.

18. The method of claim 1, wherein the nucleic acid component comprises one or more vectors that encode a gag protein targeted for secretion, a gag protein targeted for degradation, an env protein targeted for secretion, an env protein target for degration, a Pol, Nef, Tat, Vif protein targeted for degradation and a Pol, Nef, Tat protein targeted for degradation; and an IL-12 adjuvant.

19. The method of claim 18, wherein the nucleic acid component comprises Gag expression vectors 2S CATEDX and 21S MCP3p39; Env expression vectors 72S CATEenv and 73S MCP3-Env; Pol Nef Tat Vif protein expression vectors 44S CATE-PolNTV and 155S CATE-PolNT; and the protein component comprises inactive HIV particles.

20. The method of claim 19, wherein the nucleic acid component further comprises at least one expression cassette that encodes one or more polypeptides selected from the group consisting of IL-12, IL15, and granulocyte macrophage colony stimulating factor (GM-CSF).

21. The method of claim 1, wherein the nucleic acid component comprises one or more vectors that encode a gag protein a secreted gag protein, an env protein, a secreted pol protein, a pol protein targeted for degradation and a nef, tat, vif protein targeted for degradation.

22. The method of claim 21, wherein the nucleic acid component further comprises a vector encoding IL-12.

23. The method of claim 1, wherein the individual is co-immunized with the nucleic acid component and protein component at least twice.

24. The method of claim 1, wherein the nucleic acid component is administered into the muscle using electroporation.
